(19) United States
(12) Patent Application Publication
(10) Pub. No.: US 2023/0066330 A1
(43) Pub. Date: Mar. 2, 2023

(54) AN EPIGENETIC CLOCK FOR GALLIFORMES

(71) Applicant: EVONIK OPERATIONS GMBH, Essen (DE)

(72) Inventors: Günter RADDATZ, Heidelberg (DE); Frank LYKO, Hirschberg an der Bergstrasse (DE); Florian BÖHL, Neckargemünd (DE); Andreas KAPPEL, Glashütten (DE); Emeka Ignatius IGWE, München (DE); Frank THIEMANN, Nottuln (DE); Stefan PELZER, Gütersloh (DE)

(21) Appl. No.: 17/794,269

(22) PCT Filed: Jan. 22, 2021
(86) PCT No.: PCT/EP2021/051444

§ 371 Date: Jul. 21, 2022

(30) Foreign Application Priority Data
Jan. 24, 2020 (EP) 20153500.2

(51) Int. Cl. C12Q 1/6883

(52) U.S. Cl. CPC C12Q 1/6883; C12Q 2600/154; C12Q 2600/124; C12Q 1/6869

(57) ABSTRACT

The invention pertains to an in vitro method for predicting the chronological age of healthy Galliformes, the method comprising the steps of: (a.) obtaining genomic DNA from biological sample material deriving from the Galliformes subject or from the Galliformes population to be tested, (b.) determining the methylation level of a set of specific CpG sites in the genomic Galliformes DNA obtained in step (a.), and (c.) comparing the methylation levels of these CpG sites in the genomic Galliformes DNA from the sample to be tested with the methylation level of the same CpG sites from an age-correlated reference sample, thereby establishing the epigenetic age and predicting the chronological age of the subject or of the population to be tested; wherein for the set of specific CpG sites in step (b) the impact of genetic polymorphisms is eliminated by excluding CpG sites associated with single nucleotide polymorphisms, and the impact of sex-specific methylation differences on sex chromosomes is eliminated by excluding all CpG sites located on sex chromosomes.

### FIELD OF THE INVENTION

The present invention relates to a method for establishing the epigenetic age of Galliformes, and, based thereon, to a method for estimating the inflammation status in Galliformes.

### BACKGROUND OF THE INVENTION

Galliformes, such as chicken (Gallus gallus), are a significant source of commercially produced meat and eggs. Factors that influence the growth, pathogen resistance and meat quality of chicken are thus of considerable scientific and economical interest. Extensive genome-wide association studies have been conducted to elucidate the underlying genetic framework. Epigenetic modifications provide an important complement and extension to genetic variants but have remained relatively underexplored in chicken.

Animal methylomes can be highly diverse, ranging from certain insect genomes with sparse methylation patterns and only tens of thousands of methylation marks to mammalian genomes with dense methylation patterns and tens of millions of methylation marks. Until now, only little is known about the genome-wide DNA methylation patterns of non-mammalian vertebrates, and particularly of birds.

DNA methylation correlates with ageing processes and represents an epigenetic modification with a high specificity for CpG dinucleotides (5′-C-phosphate-G-3), i.e. regions of DNA where a cytosine nucleotide is followed by a guanine nucleotide in the linear sequence of bases along its 5′→3″direction. The set of genomic methylation modifications constitutes the methylome of a given cell.

Low-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described in the mouse (Stadler et al. Nature 480, 490-495 (2011)). Evolutionary conservation of LMRs beyond mammals has remained unexplored.

Age-correlated DNA methylation changes at discrete sets of CpGs in the human genome have been identified and used to predict age (Horvath, S. (2013). DNA methylation age of human tissues and cell types. Genome Biology 14:3156). These “epigenetic clocks” can estimate the DNA methylation age in specific tissues or tissue-independently and can predict mortality and time to death.

Epigenetic age is highly correlated with chronological age also respond to environmental factors that accelerate or decelerate ageing processes, resulting in substantial deviations from chronological age.

Epigenetic age acceleration (epigenetic age>chronological age) suggests that the underlying tissue ages faster than expected on the basis of chronological age, whereas a negative value (epigenetic age<chronological age, age deceleration) suggests that the tissue ages slower than would be expected. Epigenetic age acceleration is associated with a great number of age-related conditions and diseases, such as inflammatory processes.

For animal farming, performance biomarkers are particularly useful tools, as they facilitate monitoring large groups of animals and provide objective quality assurance. Galliformes, and in particular the broiler chicken represents a unique challenge for performance biomarker development, as they combine considerable economic importance with a short lifespan up to 63 days).

When it comes to welfare and performance of Galliformes, and in particular of livestock chickens, intestinal health is critically important. Enteric diseases, which are usually associated with inflammatory processes and affect the structural integrity of the gastrointestinal tract (GIT) lead to high economic losses due to reduced weight gain, poor feed conversion efficiency, increased mortality rates and greater medication costs (M'Sadeq, S. A., Wu, S., Swick, R. A. & Choct, M. (2015). Towards the control of necrotic enteritis in broiler chickens with in-feed antibiotics phasing-out worldwide. Animal Nutrition, 1, 1-11; Timbermont, L., Haesebrouck, F., Ducatelle, R. & Van Immerseel, F. (2011). Necrotic enteritis in broilers: an updated review on the pathogenesis. Avian Pathol, 40, 341-347).

Similar considerations apply for other avian species, and in particular for the species of the order of Galliformes, such as turkey, quail or pheasants.

Accordingly, new descriptive and predictive markers for biological conditions (such as inflammation of the gut) are urgently needed for controlling ongoing production processes and enabling early intervention, where necessary.

In view of the above, it was the objective of the present invention to provide robust methods for establishing the epigenetic age of Galliformes, such as chicken, with improved specificity, accuracy and precision; and to provide a method for establishing the inflammation status, respectively.

### SUMMARY OF THE INVENTION

The present invention pertains to an in vitro method for predicting the chronological age of healthy Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age and predicting the chronological age of the subject or of the population to be tested;
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

The term “healthy” in the context of the present invention refers in particular to Galliformes free of inflammatory health issues. The inventors have found that the epigenetic age of a non-inflamed Galliformes subject or of a non-inflamed Galliformes population corresponds to its chronological age, whereas deviations between epigenetic age and chronological age are indicative of inflammatory processes.

In addition, the present invention provides an in vitro method for establishing the epigenetic age of Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age of the subject or of the population to be tested; wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

Finally, the present invention relates to an in vitro method for estimating the inflammation status in Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age of the subject or of the population to be tested, and

(d.) comparing the thus-obtained epigenetic age of the subject or of the population to be tested with its actual chronological age,

wherein an epigenetic age higher than the chronological age is indicative of inflammation, and
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

### DETAILED DESCRIPTION OF THE INVENTION

The present invention provides a new epigenetic clock for Galliformes, in which CpG sites correlated with previously unrecognized confounding factors were removed. Accordingly, the new clock provides a substantially improved generalization capability and robustness.

More specifically, the inventors have identified a number of CpG (Cytosine-phosphate-Guanine) sites in the chicken (Gallus gallus) genome for which the level of DNA methylation is both tissue-specifically and tissue-independently correlated with chronological age. From these CpG sites, the impact of genetic polymorphisms is eliminated by excluding CpG sites associated with single nucleotide polymorphisms (SNPs), and/or the impact of sex-specific methylation differences on sex chromosomes is eliminated by excluding all CpG sites located on sex chromosomes. SNPs of Galliformes may be found in specific databases, such as databases, such as dbSNP (https://www.ncbi.nlm.nih.gov/snp/). Similar considerations apply for the Galliformes sex chromosomes.

In addition to the above, the methylation levels of the set of the specific CpG sites can be normalized tissue-specifically. Normalization is performed by computing for every CpG the average methylation value over all samples from the same tissue and subtracting the thus-obtained value from the value of this CpG (or, alternatively by computing for every LMR the average methylation value over all samples from the same tissue and subtracting the thus-obtained value from the value of this LMR). This normalization is necessitated by the different aging trajectories of individual tissues.

That is, measuring DNA methylation at the thus-obtained CpG sites enables determining or establishing the epigenetic age of chicken and making accurate predictions of the chronological age of chicken, respectively.

The above-described method and especially the technique of removing the confounding factors from the CpG sites is easily transferable from chicken to other Galliformes.

Prediction of Chronological Age

Based on the above findings, a new multi-tissue age predictor for Galliformes (“epigenetic clock”/“methylation clock”) has been developed.

Accordingly, the present invention provides an in vitro method for predicting the chronological age of healthy Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age and predicting the chronological age of the subject or of the population to be tested;
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

Galliformes is an order of heavy-bodied ground-feeding birds which includes turkey, grouse, chicken, ptarmigan, quail, partridge, pheasant, francolin, junglefowl and the Cracidae. This order contains five families: Phasianidae (including chicken (Gallus gallus), quail, partridges, pheasants, turkeys, peafowl and grouse), Odontophoridae, Numidiae, Cracidae and Megapodiiae.

The method according to the present invention is particularly suitable for chicken (Gallus gallus). Accordingly, one specific embodiment of the present invention is an in vitro method for predicting the chronological age of healthy chicken (Gallus gallus), the method comprising the steps of:

(a.) obtaining genomic DNA from biological sample material deriving from the chicken subject or from the chicken population to be tested,

(b.) determining the methylation level of a set of specific CpG sites in the genomic chicken DNA obtained in step (a.), and

(c.) comparing the methylation levels of these CpG sites in the genomic chicken DNA from the sample to be tested with the methylation level of the same CpG sites from an age-correlated reference sample,

thereby establishing the epigenetic age and predicting the chronological age of the subject or of the population to be tested;
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

The age-correlated reference sample serves as a control and represents an average methylation level at a pre-determined and specific chronological age.

The term “chronological age” refers to the calendar time that has passed from birth/hatch.

The epigenetic age depends on the biological state or condition of an individual or of a population and takes into account the circumstances of life (such as stress, nutrition, etc.). The terms “epigenetic age”, “methylation age”, and “biological age” have identical meanings and are used interchangeably in the context of the present application.

The term “CpG site”, “clock CpG” or “CpG location” as used in the context of the present invention refers to a CpG position that is potentially methylated. Methylation typically occurs in a CpG containing nucleic acid. The CpG containing nucleic acid may be present in, e.g. a CpG island, a CpG doublet, a promoter, an intron, or an exon of a gene or in an intergenic region. For instance, the potential methylation sites may encompass the promoter/enhancer regions of the indicated genes.

The “set of specific CpG sites in the genomic Galliformes/chicken DNA” refers to the CpG locations showing the best correlations with age.

Preferably, in addition to the above, the methylation levels of the set of the specific CpG sites in step (b) are normalized tissue-specifically.

In a preferred embodiment of the present invention, the Galliformes subject or population to be tested belongs to the species Gallus gallus and the set of specific CpG sites comprises or consists of the CpG sites indicated in Table 1.

In an alternative embodiment, the Galliformes subject or population to be tested belongs to the species Gallus gallus and the set of specific CpG sites comprises or consists of the CpG sites indicated in Table 2. In a further embodiment, the Galliformes subject or population to be tested belongs to the species Gallus gallus and the set of specific CpG sites comprises or consists of the CpG sites indicated in Table 3.

The method for predicting the chronological age of Galliformes can be used for testing individual animals and for testing a complete animal population, such as a chicken or broiler/layer flock.

The biological sample material deriving from the subject or from the population to be tested may be, for example, selected from the group consisting of body fluids, excremental material, tissue material, such as muscle tissue, gut tissue, organ tissue, skin tissue, feather material, such as quill pen, or combinations thereof. Excremental material includes gut content, fecal and cecal excrements, litter samples, as well as mixtures, solutions or suspensions thereof. An example for muscle tissue is breast (pectoralis major), examples for gut tissue are ileum and jejunum; and examples for organ tissue are spleen tissue or heart tissue. The term “litter sample” refers to mixed fecal droppings comprising residues of bedding material.

The biological sample deriving from the subject or from the population to be tested is preferably feces. Fecal sample material can be collected ante mortem. The DNA material isolated from feces contains significant amounts of gut cell DNA (mucosa).

In a particularly preferred embodiment, biological sample deriving from the subject or from the population to be tested is pooled fecal sample material deriving from a Galliformes population. Pooled fecal sample material is obtained by combining and mixing individual fecal samples.

The sample size (i.e. the number of excremental samples to be taken; each sample taken at a specific site within the animal house) has to be determined in view of the actual stocking density, i.e. with the actual number of animals belonging to the population to be tested.

In general, a minimum of 80 to 100 individual excremental samples are sufficient for most livestock chicken populations. As an example, for a broiler flock of 20000 animals, 96 individual samples are required for a confidence level of 95%.

For obtaining the pooled excremental sample material, several sampling methods may be used. In one embodiment, the pooled excremental sample is obtained by systematic grid sampling (systematic random sampling). For this method, the animal house or area in which the avian population is kept is divided in a grid pattern of uniform cells or sub-areas based on the desired number of individual excremental samples (i.e. the sample size). Then, a random sample collection site is identified within the first grid cell and a first sample is taken at said site. Finally, further samples are obtained from adjacent cells sequentially—e.g. in a serpentine, angular or zig-zag fashion—using the same relative location within each cell. A random starting point can be obtained with a dice or a random number generator. The above process may optionally be repeated for replicate samples.

Step (b.) of the in vitro method for establishing the epigenetic age of Galliformes, and in particular of chicken, may include a DNA methylation profiling process, preferably bisulfite sequencing. Therein, cytosine residues in the genomic DNA are transformed to uracil, while 5-methylcytosine residues in the genomic DNA are not transformed to uracil.

Whole genome bisulfite sequencing is a genome-wide analysis of DNA methylation based on the sodium bisulfite conversion of genomic DNA, which is then sequenced on a next-generation sequencing platform. The sequences are then re-aligned to the reference genome to determine methylation states of the CpG dinucleotides based on mismatches resulting from the conversion of unmethylated cytosines into uracil.

For example, methylation levels can be measured using the commercial Illumina™ platform.

To quantify the methylation level, various established protocols may be used to calculate the beta value of methylation, which equals the fraction of methylated cytosines in a specific location.

Step (c) may be performed with a mathematical algorithm and in particular with a statistical prediction method.

The selection of the CpGs, which define the clock, i.e. the set of specific CpG sites of step (b), may be done with a penalized regression. In this case, the evaluation of a newly sequenced test sample is done by evaluating the methylation values applying the existing regression function of the clock. In accordance therewith, a trained regression function is preferably applied in step (c).

Preferably, the Galliformes subject or population to be tested is/are broiler(s) having a life span of up to 63 days.

Determination of Epigenetic Age

The epigenetic age generally depends on the biological state or condition of an individual (or of a population).

Epigenetic age may match or mismatch with chronological age. Deviations of the epigenetic age from the chronological age are age acceleration or age deceleration.

Accordingly, epigenetic age may also be determined by comparison of the methylation levels of the methylation markers (i.e. CpG sites) in the genomic Galliformes DNA from the sample to be tested with the methylation status of the same markers (i.e. CpG sites) from an age-correlated reference sample. The term “age-correlated reference sample” is to be understood as defined above.

More specifically, the present invention provides an in vitro method for establishing the epigenetic age of Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age of the subject or of the population to be tested;
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

The method according to the present invention is particularly suitable for chicken (Gallus gallus). Accordingly, one specific embodiment of the present invention is an in vitro method for establishing the epigenetic age of Galliformes, the method comprising the steps of:

(a.) obtaining genomic DNA from biological sample material deriving from the chicken subject or from the chicken population to be tested,

(b.) determining the methylation level of a set of specific CpG sites in the genomic chicken DNA obtained in step (a.), and

(c.) comparing the methylation levels of these CpG sites in the genomic chicken DNA from the sample to be tested with the methylation level of the same CpG sites from an age-correlated reference sample,

thereby establishing the epigenetic age of the subject or of the population to be tested;
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

Preferably, in addition to the above, the methylation levels of the set of the specific CpG sites in step (b) were normalized tissue-specifically.

In a preferred embodiment of the present invention, the Galliformes subject or population to be tested belongs to the species Gallus gallus and the set of specific CpG sites comprises or consists of the CpG sites indicated in Table 1.

In an alternative embodiment, the Galliformes subject or population to be tested belongs to the species Gallus gallus and the set of specific CpG sites comprises or consists of the CpG sites indicated in Table 2. In a further embodiment, the Galliformes subject or population to be tested belongs to the species Gallus gallus and the set of specific CpG sites comprises or consists of the CpG sites indicated in Table 3.

The method for predicting the chronological age of Galliformes can be used for testing individual animals and for testing a complete animal population, such as a chicken or broiler/layer flock.

The sample material and the sampling conditions are as described above. Preferably, the biological sample material deriving from the subject or from the population to be tested is selected from the group consisting of body fluids, excremental material, tissue material, such as muscle tissue, organ tissue, such as gut tissue, skin tissue, feather material, or combinations thereof.

Step (b.) of the in vitro method for establishing the epigenetic age of Galliformes, and in particular of chicken, may include a DNA methylation profiling process, preferably bisulfite sequencing. Step (c) may be performed with a mathematical algorithm and in particular with a statistical prediction method. The selection of the CpGs, which define the clock, i.e. the set of specific CpG sites of step (b), may be done with a penalized regression. In this case, the evaluation of a newly sequenced test sample is done by evaluating the methylation values applying the existing regression function of the clock. In accordance therewith, a trained regression function is preferably applied in step (c).

Preferably, the Galliformes subject or population to be tested is/are broiler(s) having a life span of up to 63 days.

Estimating the Inflammation Status

The inventors have found that in Galliformes, and in particular in chicken (Gallus gallus), a mismatch of epigenetic and chronological age, and in particular epigenetic age acceleration (i.e. epigenetic age>chronological age) is an early indication of inflammatory processes.

Accordingly, the present invention also pertains to an in vitro method for estimating the inflammation status in Galliformes, the method comprising the steps of:

(a.) obtaining genomic DNA from biological sample material deriving from the Galliformes subject or from the Galliformes population to be tested,

(b.) determining the methylation level of a set of specific CpG sites in the genomic Galliformes DNA obtained in step (a.), and

(c.) comparing the methylation levels of these CpG sites in the genomic Galliformes DNA from the sample to be tested with the methylation level of the same CpG sites from an age-correlated reference sample,

thereby establishing the epigenetic age of the subject or of the population to be tested, and

(d.) comparing the thus-obtained epigenetic age of the subject or of the population to be tested with its actual chronological age,

wherein an epigenetic age higher than the chronological age is indicative of inflammation, wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

In one specific embodiment, the invention relates to an in vitro method for estimating the inflammation status in livestock chicken (Gallus gallus), the method comprising the steps of:

(a.) obtaining genomic DNA from biological sample material deriving from the chicken subject or from the chicken population to be tested,

(b.) determining the methylation level of a set of specific CpG sites in the genomic chicken DNA obtained in step (a.), and

(c.) comparing the methylation levels of these CpG sites in the genomic chicken DNA from the sample to be tested with the methylation level of the same CpG sites from an age-correlated reference sample,

thereby establishing the epigenetic age of the subject or of the population to be tested, and

(d.) comparing the thus-obtained epigenetic age of the subject or of the population to be tested with its actual chronological age,

wherein an epigenetic age higher than the chronological age is indicative of inflammation,
wherein for the set of specific CpG sites in step (b)
the impact of genetic polymorphisms is eliminated by excluding CpG sites associated with single nucleotide polymorphisms, and
the impact of sex-specific methylation differences on sex chromosomes is eliminated by excluding all CpG sites located on sex chromosomes.

Preferably, in addition to the above, the methylation levels of the set of the specific CpG sites in step (b) were normalized tissue-specifically.

In a preferred embodiment of the present invention, the Galliformes subject or population to be tested belongs to the species Gallus gallus and the set of specific CpG sites comprises or consists of the CpG sites indicated in Table 1.

In an alternative embodiment, the Galliformes subject or population to be tested belongs to the species Gallus gallus and the set of specific CpG sites comprises or consists of the CpG sites indicated in Table 2. In a further embodiment, the Galliformes subject or population to be tested belongs to the species Gallus gallus and the set of specific CpG sites comprises or consists of the CpG sites indicated in Table 3.

The biological sample material deriving from the subject or from the population to be tested may be, for example, selected from the group consisting of body fluids, excremental material, tissue material, such as muscle tissue, gut tissue, organ tissue, skin tissue, feather material, such as quill pen, or combinations thereof. Excremental material includes gut content, fecal and cecal excrements, litter samples, as well as mixtures, solutions or suspensions thereof. An example for muscle tissue is breast (pectoralis major), examples for gut tissue are ileum and jejunum; and examples for organ tissue are spleen tissue or heart tissue. The term “litter sample” refers to mixed fecal droppings comprising residues of bedding material.

The biological sample deriving from the subject or from the population to be tested is preferably feces. Fecal sample material can be collected ante mortem. The DNA material isolated from feces contains significant amounts of gut cell DNA (mucosa).

In a particularly preferred embodiment, biological sample deriving from the subject or from the population to be tested is pooled fecal sample material deriving from a Galliformes population, such as a chicken population. Pooled fecal sample material is obtained by combining and mixing individual fecal samples.

The sample size (i.e. the number of excremental samples to be taken; each sample taken at a specific site within the animal house) has to be determined in view of the actual stocking density, i.e. with the actual number of animals belonging to the population to be tested.

In general, a minimum of 80 to 100 individual excremental samples are sufficient for most livestock chicken populations. As an example, for a broiler flock of 20000 animals, 96 individual samples are required for a confidence level of 95%.

For obtaining the pooled excremental sample material, several sampling methods may be used. In one embodiment, the pooled excremental sample is obtained by systematic grid sampling (systematic random sampling). For this method, the animal house or area in which the avian population is kept is divided in a grid pattern of uniform cells or sub-areas based on the desired number of individual excremental samples (i.e. the sample size). Then, a random sample collection site is identified within the first grid cell and a first sample is taken at said site. Finally, further samples are obtained from adjacent cells sequentially—e.g. in a serpentine, angular or zig-zag fashion—using the same relative location within each cell. A random starting point can be obtained with a dice or a random number generator. The above process may optionally be repeated for replicate samples.

As an example for broiler flocks, excremental samples may be collected and analyzed on a daily basis during the initial growth phase (starter phase, day 5 to day 10), and/or during the enhanced growth phase (day 11 to day 18) and, optionally, also on a later stage. Alternatively, the excremental sample material, in particular fecal sample material, from the broiler flock is collected and analyzed on a daily basis starting from day 10.

Preferably, the Galliformes subject or population to be tested is/are broiler(s) having a life span of up to 63 days.

The life cycle of chicken starts with eggs taken from parent birds in the hatchery which are then incubated at a constant temperature for 21 days until the birds hatch, though at this stage the precocial chicken might be up to 72 hours old they are called one-day chicken. These chickens are separated by sexes and the female birds are kept for approx. one year for laying eggs.

The lifespan for broiler chicken is significantly shorter and varies between 21 days up to 170 days. An average US broiler is slaughtered after 47 days at a slaughter weight of 2.6 kg while in Europe the average slaughter age is at 42 days (at a weight of 2.5 kg).

Broilers are usually kept in flocks which can consist of 20.000 birds of more in one house and are fed with up to three different feed types (starter feed, grower feed and finisher feed) during this production cycle. Those feed types are adjusted to specific production phases, i.e. the initial growth phase (starter phase, day 5 to day 10), the enhanced growth phase (starting about day 11), and the finisher phase. The feeding regime also influences the methylation levels. Accordingly, also non-optimized feed may also lead to accelerated ageing (epigenetic age>chronological age).

Further, the birds are usually exposed to a number of external environmental factors, such as bacteria, viruses, parasites, diet or climate. These factors influence the outcome of a production cycle in terms of flock performance or flock uniformity and manifest in a different methylation pattern of a single bird or of a flock which may result in age acceleration that could be detected. Step b), determining the methylation level of a set of specific CpG (Cytosine-phosphate-Guanine) sites (“clock CpGs”) in the genomic Galliformes or chicken DNA, may include a DNA methylation profiling process, preferably bisulfite sequencing. Step (c) may be performed with a mathematical algorithm and in particular with a statistical prediction method.

The selection of the CpGs, which define the clock, i.e. the set of specific CpG sites of step (b), may be done with a penalized regression. In this case, the evaluation of a newly sequenced test sample is done by evaluating the methylation values applying the existing regression function of the clock. In accordance therewith, a trained regression function is preferably applied in step (c).

As shown in the above, epigenetic age is correlated with the health condition and in particular with the inflammation status of a Galliformes livestock. Accordingly, based on the methods according to the invention, necessity of therapeutic or nutritional interventions may be evaluated based thereon.

Such intervention may include providing an individualized (tailored) treatment to the individual or population tested to bring the predicted epigenetic age closer to the chronological age of the individual or population.

Further, such treatment or intervention may include feeding or administering health-promoting substances, such as zootechnical feed additives, or therapeutic agents. The term “administering” or related terms includes oral administration. Oral administration may be via drinking water, oral gavage, aerosol spray or animal feed. The term “zootechnical feed additive” refers to any additive used to affect favorably the performance of animals in good health or used to affect favorably the environment. Examples for zootechnical feed additives are digestibility enhancers, i.e. substances which, when fed to animals, increase the digestibility of the diet, through action on target feed materials; gut flora stabilizers; micro-organisms or other chemically defined substances, which, when fed to animals, have a positive effect on the gut flora; or substances which favorably affect the environment. Preferably, the health-promoting substances are selected from the group consisting of probiotic agents, prebiotic agents, botanicals, organic/fatty acids, bacteriophages and bacteriolytic enzymes or any combinations thereof.

In addition to the above, the present invention also pertains to the use of the methods disclosed herein for the development of a routine analysis tool such as real-time PCR, targeted sequencing/panel sequencing, methylated DNA immunoprecipitation as input for both, chip/array technology or methylated DNA sequencing.

Applications of the methods according to the invention are for example ((i) aiding in evaluation of the health status of Galliformes, such as chicken (ii) monitoring the progress or reoccurrence of clinical and sub-clinical disorders or (iii) studying the effects of medication, feed compounds and/or special diets on the biological age—and thus on the health status of Galliformes, such as chicken. Applications of the methods according to the present invention in particular help to avoid loss in animal performance like weight gain and feed conversion.

### EXAMPLES

Methods

Samples

Animals were stratified into four tissue (breast, ileum, spleen and jejunum) and three age (3 d, 15 d, 34 d) groups, in case of jejunum 14 d, 16 d and 35 d. From each of these 12 groups, DNA was prepared from three independent animals, resulting in 36 genomic DNA samples.

Whole-Genome Bisulfite Sequencing

Whole-genome bisulfite sequencing libraries were prepared using the Accel-NGS Methyl-Seq DNA Library Kit from Swift Biosciences. Two sequencing libraries were barcoded onto one sequencing lane. Sequencing was performed on an Illumina HiSeq X platform using a standard paired-end sequencing protocol with 105 nucleotides read length.

Read Mapping

Reads were trimmed and mapped with BSMAP 2.5 (Xi Y, Li W. 2009. BSMAP: whole genome bisulfite sequence MAPping program. BMC Bioinformatics 10:232. doi:10.1186/1471-2105-10-232.) using the Gallus gallus genome assembly version 5.0 (https://www.ebi.ac.uk/ena/data/view/GCA_000002315.3) as a reference sequence. Duplicates were removed using the Picard tool (http://broadinstitute.github.io/picard). Methylation ratios were determined using a Python script (methratio.py) distributed together with the BSMAP package by dividing the number of reads having a methylated CpG at a certain genomic position by the number of all reads covering this position.

Normalization and SNP Filtering of the Methylation Data

All CpGs which are listed as SNPs in the database dbSNP (https://www.ncbi.nlm.nih.gov/snp/) for the Gallus gallus genome were filtered out. All CpGs and LMRs mapping to the Galliformes sex Chromosomes W and Z were filtered out and removed from the data sets. For the genome-wide clock, the analysis was restricted to CpGs that showed a strand specific coverage of greater than 10 in every of the sequenced samples, resulting in a set of 257,913 CpGs. Then the data were normalized by computing for every CpG the average methylation value over all samples from the same tissue and subtracted this value from the methylation value of this CpG. For the LMR clock, the analysis was restricted to CpGs within low-methylated regions that showed a strand specific coverage of greater than 5 in every of the sequenced samples, resulting in a set of 67,651 LMRs. The average methylation values of these LMRs were computed and normalized by computing for every LMR the average value over all samples from the same tissue and subtracting this value from the value of this LMR.

Establishment of a Chicken DNA Methylation Clock

Then a penalized regression model (implemented in the R package glmnet [https://cran.r-project.org/web/packages/glmnet/]) was applied to regress the chronological age of the animals on the normalized methylation values of the CpG probes. In the case of the LMR clock a penalized regression model was applied to regress the chronological age of the animals on the normalized average methylation values of the LMRs.

Results

Genome-Wide Clock

The alpha parameter of glmnet was varied in a range between 0 and 1 and chosen as 0.7 (elastic net regression), because this value led to a fit that was close to the best fit and a manageable amount of CpGs. The lambda value was chosen using cross-validation on the training data as 0.4016. This identified a set of 45 CpGs together with corresponding beta values, which define the weights for these CpGs used in the chicken methylation clock. The mean squared error of 6-fold crossvalidation using the values of 0.7 for alpha and 0.4016 for lambda was 11.538. This indicates that a new sample can be predicted with an error of about 3.4 days. In order to apply the clock to a new sample the methylation ratios of this sample at the 45 clock CpGs have to be provided and the command predict.cv of the package glmnet with the trained clock has to be performed.

FIG. 1 shows the mean squared error of a trained clock for given alpha at value of lambda leading to the minimal error.

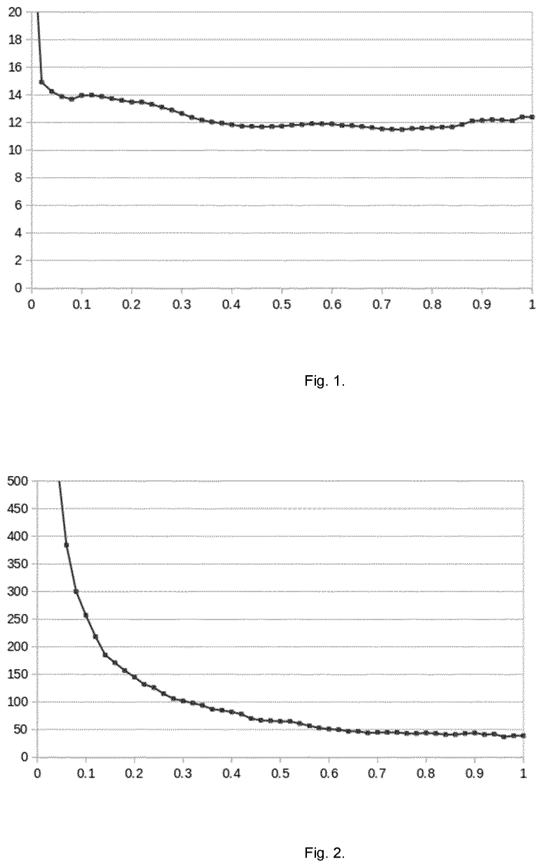

FIG. 2 shows the number of CpGs for given alpha at value of lambda leading to the minimal error.

TABLE 1
Clock CpGs (genome-wide methylation, alpha = 0.7,
lambda = 0.4016, #CpG's: 45).

| ID | chrom | position | weight | Ileum ¹ | Spleen ¹ | Breast ¹ | Jejunum ¹ |
|---|---|---|---|---|---|---|---|
| 1 | chr1 | 26806096 | −0.333 | 0.636 | 0.475 | 0.464 | 0.64 |
| 2 | chr1 | 27051068 | −1.207 | 0.363 | 0.124 | 0.445 | 0.235 |
| 3 | chr1 | 79412910 | −3.879 | 0.467 | 0.438 | 0.573 | 0.414 |
| 4 | chr1 | 193007724 | −0.894 | 0.504 | 0.181 | 0.398 | 0.44 |
| 5 | chr2 | 84879641 | 2.595 | 0.381 | 0.665 | 0.191 | 0.415 |
| 6 | chr2 | 139780944 | −0.004 | 0.32 | 0.198 | 0.053 | 0.182 |
| 7 | chr3 | 9654592 | −2.179 | 0.503 | 0.328 | 0.698 | 0.589 |
| 8 | chr3 | 23119819 | −2.285 | 0.282 | 0.251 | 0.31 | 0.292 |
| 9 | chr3 | 32240754 | 2.209 | 0.256 | 0.244 | 0.148 | 0.264 |
| 10 | chr3 | 55893779 | −3.285 | 0.528 | 0.563 | 0.673 | 0.564 |
| 11 | chr3 | 55933564 | −0.301 | 0.335 | 0.302 | 0.649 | 0.165 |
| 12 | chr4 | 20608622 | −0.825 | 0.547 | 0.512 | 0.554 | 0.728 |
| 13 | chr4 | 48345505 | 0.468 | 0.285 | 0.435 | 0.239 | 0.304 |
| 14 | chr4 | 70292571 | −0.001 | 0.254 | 0.235 | 0.561 | 0.332 |
| 15 | chr5 | 1942965 | 3.015 | 0.268 | 0.532 | 0.178 | 0.322 |
| 16 | chr5 | 1942982 | 2.248 | 0.334 | 0.562 | 0.174 | 0.397 |
| 17 | chr5 | 12844701 | −0.238 | 0.583 | 0.435 | 0.711 | 0.691 |
| 18 | chr5 | 16850281 | 1.412 | 0.651 | 0.784 | 0.654 | 0.723 |
| 19 | chr5 | 17507391 | −3.468 | 0.261 | 0.197 | 0.115 | 0.351 |
| 20 | chr5 | 39037892 | 1.739 | 0.476 | 0.506 | 0.379 | 0.61 |
| 21 | chr5 | 54227250 | −1.625 | 0.225 | 0.358 | 0.361 | 0.28 |
| 22 | chr5 | 58662889 | 5.718 | 0.46 | 0.621 | 0.364 | 0.503 |
| 23 | chr6 | 5240214 | −0.287 | 0.262 | 0.317 | 0.196 | 0.213 |
| 24 | chr6 | 7819244 | 4.26 | 0.209 | 0.511 | 0.234 | 0.188 |
| 25 | chr6 | 12024016 | −2.447 | 0.662 | 0.24 | 0.575 | 0.515 |
| 26 | chr6 | 12065954 | 1.12 | 0.286 | 0.388 | 0.249 | 0.325 |
| 27 | chr7 | 9815074 | −5.1 | 0.726 | 0.46 | 0.738 | 0.655 |
| 28 | chr7 | 11137846 | −0.002 | 0.367 | 0.286 | 0.587 | 0.326 |
| 29 | chr7 | 14040077 | −1.945 | 0.431 | 0.309 | 0.357 | 0.366 |
| 30 | chr7 | 21995171 | −2.653 | 0.192 | 0.057 | 0.244 | 0.137 |
| 31 | chr7 | 30586853 | 0.837 | 0.335 | 0.391 | 0.176 | 0.501 |
| 32 | chr8 | 3444574 | 1.024 | 0.255 | 0.654 | 0.388 | 0.256 |
| 33 | chr8 | 8196471 | 0.618 | 0.56 | 0.802 | 0.691 | 0.565 |
| 34 | chr8 | 18912606 | −1.112 | 0.442 | 0.333 | 0.599 | 0.542 |
| 35 | chr8 | 27250408 | −0.755 | 0.473 | 0.413 | 0.394 | 0.735 |
| 36 | chr10 | 20035839 | −0.002 | 0.251 | 0.14 | 0.142 | 0.234 |
| 37 | chr11 | 7627454 | 0.396 | 0.593 | 0.601 | 0.222 | 0.672 |
| 38 | chr14 | 9143159 | −3.085 | 0.519 | 0.34 | 0.564 | 0.355 |
| 39 | chr14 | 9143204 | −2.843 | 0.678 | 0.401 | 0.615 | 0.388 |
| 40 | chr15 | 201524 | 6.892 | 0.596 | 0.634 | 0.3 | 0.559 |
| 41 | chr15 | 8945553 | −13.223 | 0.766 | 0.724 | 0.87 | 0.542 |
| 42 | chr17 | 1673086 | −0.441 | 0.616 | 0.305 | 0.472 | 0.669 |
| 43 | chr19 | 7327224 | 5.149 | 0.657 | 0.492 | 0.266 | 0.648 |
| 44 | chr23 | 172291 | −0.279 | 0.646 | 0.538 | 0.562 | 0.479 |
| 45 | chr23 | 5568087 | −1.692 | 0.277 | 0.183 | 0.18 | 0.255 |

Intercept of linear model equation found by glmnet: 17.365
¹ Correction factors of the different tissues. The respective value has to be subtracted.

LMR Clock

### Example 1

The alpha parameter of glmnet was varied in a range between 0 and 1 and chosen as 0.84 (elastic net regression), because this value led to a fit that was close to the best fit and a manageable amount of LMRs. The lambda value was chosen using cross-validation on the training data as 0.3194. This identified a set of 39 LMRs together with corresponding beta values, which define the weights for these LMRs used in the chicken methylation clock. The mean squared error of 6-fold crossvalidation using the values of 0.84 for alpha and 0.3194 for lambda was 13.4831. This indicates that a new sample can be predicted with an error of about 3.7 days. In order to apply the clock to a new sample the methylation ratios of this sample at the 39 clock LMRs have to be provided and the command predict.cv of the package glmnet with the trained clock has to be performed.

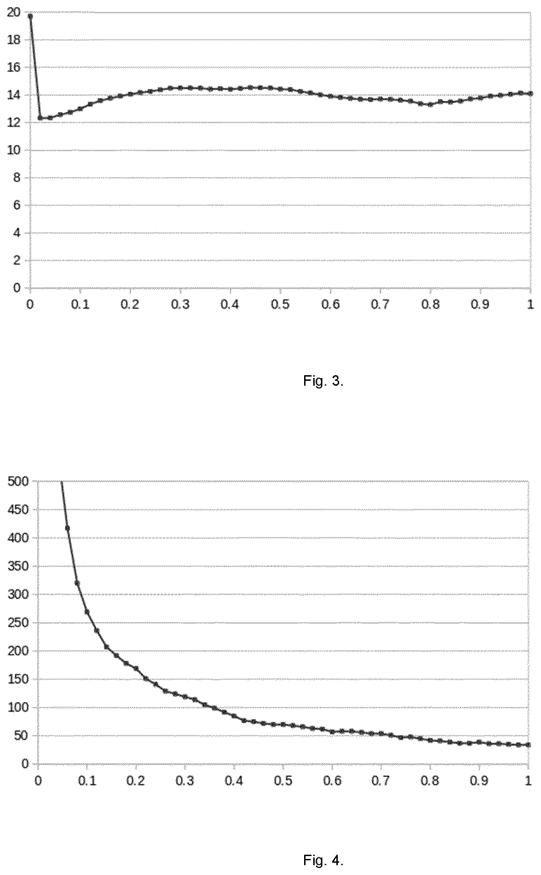

FIG. 3 shows the mean squared error of a trained clock for given alpha at value of lambda leading to the minimal error.

FIG. 4 shows the number of LMRs for given alpha at value of lambda leading to the minimal error.

TABLE 2
Clock CpGs (LMR methylation, alpha = 0.84, lambda = 0.3194, #LMR's: 39).

| ID | chrom | start | end | weight | Ileum ¹ | Spleen ¹ | Breast ¹ | Jejunum ¹ |
|---|---|---|---|---|---|---|---|---|
| 1 | chr1 | 44395372 | 44398932 | −11.474 | 0.085 | 0.119 | 0.087 | 0.111 |
| 2 | chr1 | 83295508 | 83295820 | 3.676 | 0.277 | 0.463 | 0.204 | 0.305 |
| 3 | chr1 | 194750612 | 194750882 | 1.159 | 0.09 | 0.199 | 0.071 | 0.101 |
| 4 | chr2 | 8123576 | 8124320 | 3.335 | 0.179 | 0.168 | 0.113 | 0.279 |
| 5 | chr2 | 31316252 | 31316368 | 11.63 | 0.129 | 0.087 | 0.08 | 0.111 |
| 6 | chr2 | 35582600 | 35584144 | 12.066 | 0.305 | 0.357 | 0.341 | 0.317 |
| 7 | chr2 | 42878428 | 42879088 | −1.381 | 0.479 | 0.245 | 0.336 | 0.44 |
| 8 | chr2 | 63925292 | 63925632 | 7.773 | 0.086 | 0.321 | 0.117 | 0.115 |
| 9 | chr2 | 81161918 | 81161974 | 3.276 | 0.234 | 0.491 | 0.269 | 0.241 |
| 10 | chr2 | 91174539 | 91175128 | −28.595 | 0.235 | 0.262 | 0.181 | 0.238 |
| 11 | chr2 | 103673926 | 103674122 | −1.539 | 0.215 | 0.104 | 0.191 | 0.174 |
| 12 | chr3 | 77360372 | 77360404 | 1.67 | 0.152 | 0.263 | 0.1 | 0.199 |
| 13 | chr5 | 839710 | 840094 | 5.314 | 0.231 | 0.328 | 0.145 | 0.233 |
| 14 | chr5 | 1942054 | 1942842 | 1.067 | 0.325 | 0.414 | 0.23 | 0.349 |
| 15 | chr5 | 28482294 | 28482418 | 0.767 | 0.113 | 0.304 | 0.09 | 0.264 |
| 16 | chr5 | 39059306 | 39059368 | 3.441 | 0.025 | 0.068 | 0.028 | 0.058 |
| 17 | chr6 | 8416238 | 8416588 | 21.541 | 0.13 | 0.2 | 0.09 | 0.16 |
| 18 | chr7 | 5169488 | 5169670 | 2.308 | 0.232 | 0.23 | 0.244 | 0.213 |
| 19 | chr7 | 17839660 | 17839728 | −5.446 | 0.685 | 0.445 | 0.579 | 0.617 |
| 20 | chr9 | 23812488 | 23812678 | 4.227 | 0.155 | 0.382 | 0.185 | 0.151 |
| 21 | chr11 | 675297 | 675546 | −1.501 | 0.316 | 0.329 | 0.59 | 0.346 |
| 22 | chr12 | 1688020 | 1688132 | 0.37 | 0.163 | 0.359 | 0.166 | 0.213 |
| 23 | chr12 | 6875861 | 6876152 | −0.25 | 0.301 | 0.084 | 0.212 | 0.277 |
| 24 | chr12 | 10983288 | 10984278 | −0.007 | 0.258 | 0.294 | 0.303 | 0.225 |
| 25 | chr12 | 16248174 | 16248357 | −1.758 | 0.598 | 0.583 | 0.819 | 0.317 |
| 26 | chr13 | 13146982 | 13147888 | −17.978 | 0.167 | 0.113 | 0.13 | 0.179 |
| 27 | chr13 | 16017638 | 16017826 | −0.017 | 0.155 | 0.224 | 0.199 | 0.14 |
| 28 | chr13 | 16716158 | 16716440 | −0.034 | 0.153 | 0.273 | 0.147 | 0.18 |
| 29 | chr14 | 4137808 | 4137912 | −0.166 | 0.259 | 0.137 | 0.22 | 0.215 |
| 30 | chr15 | 8945392 | 8945554 | −8.922 | 0.493 | 0.464 | 0.727 | 0.324 |
| 31 | chr17 | 2483692 | 2483848 | 8.025 | 0.142 | 0.286 | 0.097 | 0.204 |
| 32 | chr17 | 3822992 | 3823290 | 2.947 | 0.207 | 0.512 | 0.206 | 0.228 |
| 33 | chr17 | 10211804 | 10212170 | −3.233 | 0.099 | 0.087 | 0.189 | 0.08 |
| 34 | chr20 | 2469403 | 2470309 | −4.959 | 0.173 | 0.273 | 0.253 | 0.262 |
| 35 | chr20 | 10704150 | 10704244 | −2.422 | 0.216 | 0.137 | 0.169 | 0.195 |
| 36 | chr20 | 11718629 | 11718916 | 3.151 | 0.149 | 0.379 | 0.23 | 0.201 |
| 37 | chr23 | 2763708 | 2763780 | 2.721 | 0.331 | 0.61 | 0.428 | 0.366 |
| 38 | chr23 | 5159782 | 5159918 | −2.9 | 0.283 | 0.171 | 0.309 | 0.228 |
| 39 | chr28 | 2874382 | 2874447 | 0.005 | 0.369 | 0.328 | 0.322 | 0.327 |

Intercept of linear model equation found by glmnet: 17.411
¹ Correction factors of the different tissues. The respective value has to be subtracted.

### Example 2

The alpha value was varied in a range between 0 and 1 and chosen as 0.9 (elastic net regression). This identified a set of 32 LMRs together with corresponding beta values, which define the weights for these LMRs used in the chicken methylation clock (Tab. 3).

TABLE 3
Clock LMRs (alpha = 0.9, lambda = 0.3147).

| ID | chrom | start | end | weight | ileum | spleen | breast | jejunum |
|---|---|---|---|---|---|---|---|---|
| 1 | chr1 | 3310966 | 3311076 | 5.106 | 0.089 | 0.117 | 0.048 | 0.108 |
| 2 | chr1 | 13486724 | 13487721 | −1.078 | 0.421 | 0.180 | 0.224 | 0.424 |
| 3 | chr1 | 77403928 | 77404268 | 5.291 | 0.106 | 0.160 | 0.040 | 0.183 |
| 4 | chr1 | 131728204 | 131729184 | −6.235 | 0.407 | 0.363 | 0.318 | 0.197 |
| 5 | chr1 | 135369614 | 135369882 | −1.194 | 0.436 | 0.184 | 0.403 | 0.419 |
| 6 | chr1 | 165806748 | 165806816 | −0.009 | 0.477 | 0.527 | 0.844 | 0.542 |
| 7 | chr2 | 31315302 | 31315823 | 0.961 | 0.148 | 0.099 | 0.104 | 0.200 |
| 8 | chr2 | 31316250 | 31316368 | 15.824 | 0.129 | 0.087 | 0.059 | 0.111 |
| 9 | chr2 | 91174537 | 91175128 | −26.554 | 0.235 | 0.262 | 0.188 | 0.238 |
| 10 | chr4 | 1489570 | 1490794 | −8.003 | 0.176 | 0.149 | 0.158 | 0.214 |
| 11 | chr4 | 8453114 | 8454528 | 3.325 | 0.159 | 0.524 | 0.316 | 0.211 |
| 12 | chr4 | 31342294 | 31342536 | 0.228 | 0.638 | 0.574 | 0.638 | 0.640 |
| 13 | chr5 | 839708 | 840094 | 2.227 | 0.231 | 0.328 | 0.153 | 0.233 |
| 14 | chr5 | 1942052 | 1942842 | 2.613 | 0.325 | 0.414 | 0.204 | 0.349 |
| 15 | chr5 | 39059304 | 39059368 | 0.307 | 0.025 | 0.068 | 0.024 | 0.058 |
| 16 | chr5 | 52951604 | 52951808 | 2.676 | 0.070 | 0.148 | 0.024 | 0.091 |
| 17 | chr6 | 8416236 | 8416588 | 12.930 | 0.130 | 0.200 | 0.099 | 0.160 |
| 18 | chr8 | 13056204 | 13056776 | 4.557 | 0.142 | 0.269 | 0.122 | 0.150 |
| 19 | chr9 | 23812486 | 23812678 | 6.756 | 0.155 | 0.382 | 0.179 | 0.151 |
| 20 | chr11 | 675295 | 675546 | −3.678 | 0.316 | 0.329 | 0.638 | 0.346 |
| 21 | chr12 | 9433040 | 9433568 | 9.905 | 0.406 | 0.351 | 0.132 | 0.409 |
| 22 | chr12 | 16248172 | 16248357 | −0.539 | 0.598 | 0.583 | 0.815 | 0.317 |
| 23 | chr13 | 13146980 | 13147888 | −10.892 | 0.167 | 0.113 | 0.135 | 0.179 |
| 24 | chr13 | 16716156 | 16716440 | −0.540 | 0.153 | 0.273 | 0.166 | 0.180 |
| 25 | chr14 | 4137806 | 4137912 | −6.589 | 0.259 | 0.137 | 0.232 | 0.215 |
| 26 | chr15 | 8945390 | 8945554 | −3.262 | 0.493 | 0.464 | 0.741 | 0.324 |
| 27 | chr18 | 2358384 | 2359684 | −2.706 | 0.448 | 0.368 | 0.364 | 0.472 |
| 28 | chr19 | 9052179 | 9052244 | −9.309 | 0.601 | 0.295 | 0.258 | 0.523 |
| 29 | chr20 | 11718627 | 11718916 | 20.167 | 0.149 | 0.379 | 0.193 | 0.201 |
| 30 | chr23 | 5568088 | 5568140 | −2.259 | 0.402 | 0.290 | 0.436 | 0.439 |
| 31 | chr25 | 1101298 | 1101396 | −0.093 | 0.493 | 0.267 | 0.204 | 0.416 |
| 32 | chr26 | 4608324 | 4608370 | 2.441 | 0.163 | 0.416 | 0.228 | 0.203 |

Intercept of linear model equation found by glmnet: 17.345
Correction factors are indicated for different tissues. For correction, the corresponding value has to be subtracted.

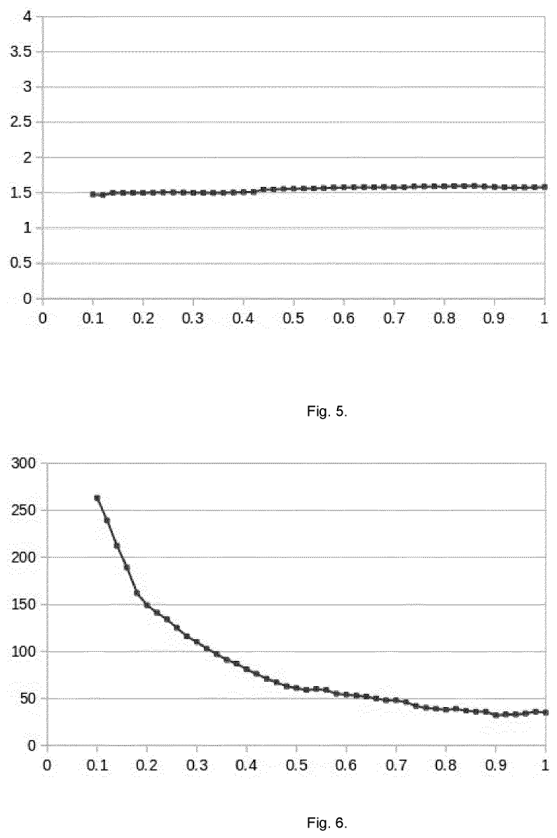

FIG. 5 shows the root mean squared error of a trained clock for given alpha at value of lambda leading to the minimal error.

FIG. 6 shows the number of LMRs for given alpha at value of lambda leading to the minimal error.

Age Prediction in Breast Tissue from a Completely Independent Validation Dataset:

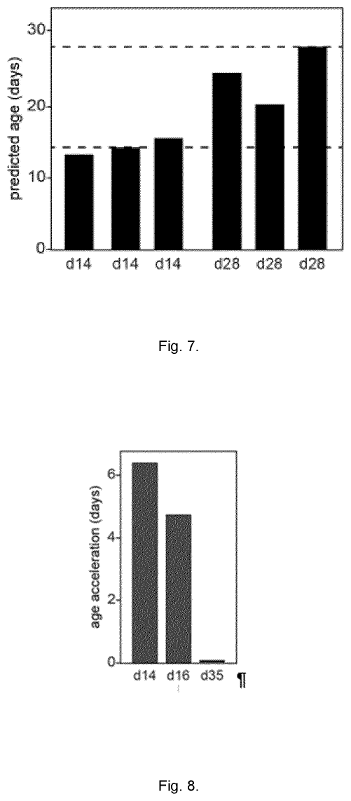

In order to validate the LMR clock, whole-genome bisulfite sequencing of 6 samples (breast) in two age groups (14 and 28 days) from a completely independent animal trial was performed. Age prediction showed a root mean square error of 2.7 days and 3.8 days, respectively, which is consistent with the prediction error obtained after cross-validation. Results are visualized in FIG. 7.

Age Acceleration as a Marker for Inflammatory Processes

Birds were injected of either CpG or the control GpC on the day after hatching and on days 13-16, 27-30, and 34-35. Jejunal tissues were collected and from samples of days 14, 16 and 35 the respective genomic DNA was isolated with a standard protocol for Whole Genome Bisulfite Sequencing.

Analysis of jejunum samples showed a pronounced and highly consistent age acceleration, in particular at days 14 and 16 (FIG. 8). A control group was injected with the non-inflammatory agent GpC and did not respond at all.

1-15. (canceled)

16. An in vitro method for predicting the chronological age of healthy Galliformes, the method comprising the steps of:
(a) obtaining genomic DNA from biological sample material derived from the Galliformes subject or from the Galliformes population to be tested;
(b) determining the methylation level of a set of specific CpG sites in the genomic Galliformes DNA obtained in step (a); and
(c) comparing the methylation levels of the CpG sites of step (b) with the methylation level of the same CpG sites from an age-correlated reference sample, thereby establishing the epigenetic age and predicting the chronological age of the subject or of the population to be tested;
and wherein, for the determination in step (b):
the impact of genetic polymorphisms is eliminated by excluding CpG sites associated with single nucleotide polymorphisms; and
the impact of sex-specific methylation differences on sex chromosomes is eliminated by excluding all CpG sites located on sex chromosomes.

17. The method of claim 16, wherein the methylation levels of the set of specific CpG sites in step (b) were normalized tissue-specifically.

18. The method of claim 16, wherein the Galliformes subject or population to be tested belongs to the species Gallus gallus and the set of specific CpG sites comprises the CpG sites indicated in Table 1.

19. The method of claim 16, wherein the Galliformes subject or population to be tested belongs to the species Gallus gallus and the set of specific CpG sites consists of the CpG sites indicated in Table 1.

20. The method of claim 16, wherein the Galliformes subject or population to be tested belongs to the species Gallus gallus and the set of specific CpG sites comprises the CpG sites indicated in Table 2.

21. The method of claim 16, wherein the Galliformes subject or population to be tested belongs to the species Gallus gallus and the set of specific CpG sites consists of the CpG sites indicated in Table 2.

22. The method of claim 16, wherein the Galliformes subject or population to be tested belongs to the species Gallus gallus and the set of specific CpG sites comprises the CpG sites indicated in Table 3.

23. The method of claim 16, wherein the Galliformes subject or population to be tested belongs to the species Gallus gallus and the set of specific CpG sites consists of the CpG sites indicated in Table 3.

24. The method of claim 16, wherein the biological sample material deriving from the subject or from the population to be tested is selected from the group consisting of: body fluids, excremental material, tissue material, feather material, and combinations thereof.

25. The method of claim 16, wherein step (b) comprises a DNA methylation profiling process.

26. The method of claim 16, wherein the Galliformes subject or population to be tested is/are broiler(s) having a life span of up to 63 days.

27. An in vitro method for estimating the inflammation status in Galliformes, the method comprising the steps of:
(a) obtaining genomic DNA from biological sample material deriving from the Galliformes subject or from the Galliformes population to be tested;
(b) determining the methylation level of a set of specific CpG sites in the genomic Galliformes DNA obtained in step (a);
(c) comparing the methylation levels of the CpG sites of step (b) with the methylation level of the same CpG sites from an age-correlated reference sample, thereby establishing the epigenetic age and predicting the chronological age of the subject or of the population to be tested; and
(d) comparing the epigenetic age of the subject or of the population to be tested as determined by steps (a)-(c) with its actual chronological age, wherein an epigenetic age higher than the chronological age is indicative of inflammation;
and wherein, for the determination in step (b):
the impact of genetic polymorphisms is eliminated by excluding CpG sites associated with single nucleotide polymorphisms; and
the impact of sex-specific methylation differences on sex chromosomes is eliminated by excluding all CpG sites located on sex chromosomes.

28. The method of claim 27, wherein the methylation levels of the set of specific CpG sites in step (b) were normalized tissue-specifically.

29. The method of claim 27, wherein the Galliformes subject or population to be tested belongs to the species Gallus gallus and the set of specific CpG sites comprises the CpG sites indicated in Table 1.

30. The method of claim 27, wherein the Galliformes subject or population to be tested belongs to the species Gallus gallus and the set of specific CpG sites consists of the CpG sites indicated in Table 1.

31. The method of claim 27, wherein the Galliformes subject or population to be tested belongs to the species Gallus gallus and the set of specific CpG sites comprises the CpG sites indicated in Table 2.

32. The method of claim 27, wherein the Galliformes subject or population to be tested belongs to the species Gallus gallus and the set of specific CpG sites consists the CpG sites indicated in Table 2.

33. The method of claim 27, wherein the biological sample material deriving from the subject or from the population to be tested is selected from the group consisting of: body fluids, excremental material, tissue material, feather material, or combinations thereof.

34. The method of claim 27, wherein step (b) comprises a DNA methylation profiling process.

35. The method of claim 27, wherein the Galliformes subject or population to be tested is/are broiler(s) having a life span of up to 63 days.
